(19) United States
(12) Patent Application Publication
(10) Pub. No.: US 2015/0088433 A1
(43) Pub. Date: Mar. 26, 2015

(54) AUTOMATION IN GASEOUS SAMPLE TESTS

(71) Applicant: Grace C. Feng-Tang, Sugar Land, TX (US)

(72) Inventor: Grace C. Feng-Tang, Sugar Land, TX (US)

(73) Assignee: Applied Lab Automation, Houston, TX (US)

(21) Appl. No.: 14/037,059

(22) Filed: Sep. 25, 2013

(51) Int. Cl. G01N 33/00; G06F 17/16

(52) U.S. Cl. CPC G01N 33/0006; G01N 33/0062; G06F 17/16; G01N 2033/0068
USPC 702/24; 702/100

(57) ABSTRACT

The present invention is related to an apparatus facilitating the automation of gaseous sample analysis and the method thereof. Specifically, the present invention is related to the apparatus that is capable of detecting and converting a reading from a first matrix to a second matrix, thus simplifies a calibration process required for the gaseous sample analysis and the method thereof.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

The present invention is related to the field of automatized gaseous sample analysis. More specifically, the present invention is related to the field of simplifying calibration proc3sses for the gaseous sample analysis

When performing gaseous sample analyses such as impurity analysis, automation can be used to save time and improve efficiency. However, analyzers that are used to perform portions of the gaseous sample analysis may have a matrix effect. A matrix is referred to so-called “background gas” suspended within the gaseous samples, including but not limited to: air, Nitrogen and Argon. The matrix effect is referred to a disturbed baseline which is caused by the matrix and must be corrected by a calibration process before or during the gaseous sample analysis.

Thus, theoretically, in each of the analyzer involved in the gaseous sample analysis, a baseline must be compensated as the analyzer must be calibrated against the matrix effect caused by the background gas. Generally, each matrix effect in each of the analyzer should be individually calibrated against and compensated for. Repeating the calibration process to each of the matrix effect may result in a time-consuming and labor-intensive task. Nevertheless, said repeating process is usually carried out manually, thus the gaseous sample analysis may require human intervention instead of being a highly automatic process. This is especially true when the status of the background gases in the gaseous sample is subject to constant change, or the gaseous samples are subject to multiple analyses using a plurality of different analyzers.

Manufacturers of the analyzers are aware of the phenomena described hereabove and often provide lists of correction factors for the gaseous sample analysis when the matrix effects are expected. Some manufacturers go further and have menu-selectable correction factors available within their analyzers. Those strategies work well for the correction factors that are simple multipliers, as in the case of diffusivity coefficient in trace oxygen analysis.

Thus, under certain conditions, the presence of the matrix effect may be corrected by using the correction factor such as a k factor to compensate for the effect. The k factor refers to any scale adjustment factor that compensates for the matrix. Its application allows the analyzers to be calibrated in a first matrix or a first background gas, and accurately analyze in the gaseous sample in a second matrix or a second background gas.

For example, the Servomex Trace Oxygen Analyzer Df 310E is one of the analyzers that use the application of the k factor (known as “Gas Scaling Factor”). It applies the k factor to correct for the background gas that differs from those calibrated against.

However, not all the matrix effects could be corrected and compensated for by using the k factor. The matrix effect can be more complicated in some cases wherein the corrections require much advanced correction techniques than a simple scalar multiplication, or the k factor.

For example, in some cases the actual result in one matrix describes a linear curve whose slope and intercept depend directly on what the matrix in which the gaseous sample is suspended. In said cases the data must undergo a linear transformation to convert it from one matrix to another.

In case where the analyzer having said matrix effect is required to complete the gaseous sample analysis, its calibration may be manually handled.

For example, the Baseline Mocon 9000, THC analyzer cannot be correctable by the application of the k factor. Thus, even if said analyzer belongs to part of an automatic analysis system, the automatic process of the system must be stopped to allow for the calibration process of the non-k factor correctable analyzer.

Moreover, in order to calibrate said analyzer, at least two standards shall be tested per matrix. The standards tested are generally a Span Gas Standard and Zero Standard. The Span Gas standard will contain a contaminant of interest at the desired level in the like background gas as the candidate sample. The Zero Standard will be comprised of the like background gas to the candidate sample with no or very little contaminant present. Thus, multiple sets of calibrating standards to support each matrix analyzed are required.

In most gaseous sample analysis the procedure may require the analyzers having both k-factor correctable and non-k-factor correctable matrix effect. Therefore, automation is impeded as each analyzer having the non-k-factor correctable matrix effect may require the automation process to be stopped in order to allow the calibration.

Therefore, there is a need to develop a method to simplify and automatize the calibration process of the gaseous sample analysis when both k-factor correctable and non-k-factor correctable matrix effects are involved. Especially, there is a need to develop a method to allow one calibration to one matrix, and apply the results of the calibrations to all the matrixes having the matrix effects, either k-factor correctable or non-k-factor correctable.

### BRIEF SUMMARY OF THE INVENTION

The present invention is related to an apparatus that performs the gaseous sample analysis at a high degree of automation and its method thereof. According to the present invention, the apparatus collects the data from all the analyzers involved in the gaseous sample analysis, populates the data where the sample is associated by species, lot identifiers and/or operator identifications, etc., and records the data over an operator selectable time range that is graphically displayed. Specifically, the present invention further comprises correction factors and correction equations for converting the matrix effects and a method of applying said correction factors and/or equations. The correction factors and equations allow the data to be converted from one matrix to another.

Therefore, in one embodiment of the present invention, the apparatus comprises: means of connections to the analyzers; a calculation device that applies the correction factors and the correction equations to the data; a storage device that stores the data; and, a display device that graphically displays the data.

In yet another embodiment, the present invention discloses a method of the gaseous sample analysis by applying the correction factors and the correction equations comprising the following steps:
1. Calibrating against a first matrix;
2. Determining whether there is at least one matrix effect in the gaseous sample;
3. If yes, determining whether the correction factor could be apply to convert a reading from a second matrix to the first matrix;
4. If yes, applying the correction factor to a second reading from the second matrix to convert said reading to a first reading from the first matrix;
5. If no, applying the correction equation to the second reading from the second matrix effect to convert said reading to a first reading from the first matrix.

In one embodiment, the first matrix could have no matrix effect.

In one embodiment, the present invention collects and calculates the data from the analysis in real-time. In a preferred embodiment, the present invention requires only one calibration to one matrix during the gaseous sample analysis. The calibrations to other matrixes may be eliminated since the readings from the other matrixes could be converted to the readings from the sole calibrated matrix by applying the correction factors and/or the correction equations.

Thus, according to one embodiment, the present invention is advantageous in the following aspects: (1), it allows the gaseous sample analysis to be performed at a high level of automation; (2), it reduces the labor and time costs by simplifying the calibration process.

### BRIEF DESCRIPTION OF THE SEVERAL VIEWS OF THE DRAWINGS

FIG. 1 is a flowchart diagram illustrating a method according to embodiments of the present invention.

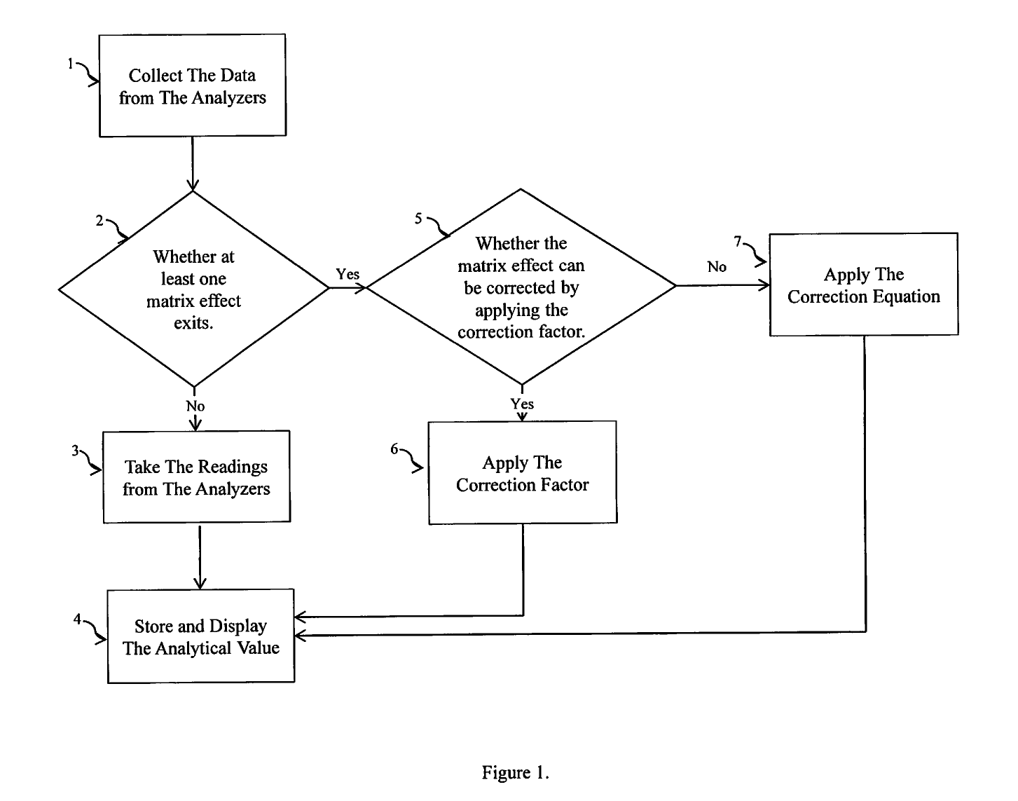

FIG. 2 is the diagram illustrating a first calibration curve and a first matrix equation using Methane as the gaseous sample with Nitrogen as the background gas.

FIG. 3 is the diagram illustrating a second calibration curve and a second matrix equation using Methane as the gaseous sample with Helium as the background gas.

Table 1 is the readings and concentrations of Methane samples with Nitrogen as the background gas.

Table 2 is the converted readings and concentrations of Methane samples with Helium as the background gas.

### DETAILED DESCRIPTION OF THE INVENTION

The present invention is related to the apparatus for the gaseous sample analysis wherein the data from the analyzers could be collected, processed and presented. Specially, the present invention is related to the apparatus and the method thereof that simplify the calibration process for the gaseous sample analysis by applying the correction factors and/or the correction equations, thus one calibration to one of the analyzers may satisfy the calibration requirement for all the analyzers involved in the whole gaseous sample analysis.

The apparatus consists of the connection means, the calculation device, the storage device and the display device. The connection means connects the apparatus with the analyzers, which allow the data to be transmitted between the apparatus and the analyzers. The connection means could be any device known in the art that performs the above function. For example, the connection means could be any physical cables and adaptors capable of transmitting digital or analog signals. For another example, the connection means could be any wireless connections between the analyzers and the apparatus which may transmit digital signals in between the two.

The calculation device, the storage device and the display device could be any device known in the art functions in data calculation, data storage and data graphical display, respectively. For example, the calculation device could be a hardware, a software or a computer programming; the storage device could be a hard drive or other memory storage devices; and the display device could be a monitor. For another example, said three devices could be independent to each other, or be included in a computer system which at least consists of a CPU, a memory device such as a hard drive or a memory card, and a screen.

Thus, in one embodiment, the gaseous sample is processed by the analyzers. A first step is to calibrate one of the analyzers connected to the apparatus and to be used in the gaseous sample test against one matrix. The calibration may be performed using methods provided by the manufacturer of the analyzer or any methods known in the art. A first calibration curve and a first matrix equation are obtained by the calibration. Alternatively, the first calibration curve and the first matrix equation may be provided by the manufacturer of the analyzer or documented in references known in the art. After the calibration, the gaseous sample is processed by the calibrated analyzer and the data including a reading and an equal-molar concentration of a first analyte was collected and stored in the apparatus (1).

A second step is to determine whether the gaseous sample analysis has the matrix effect. (2) The matrix effect is determined from the device externally. The matrix effect will be evident if the response to the first analyte produces a different reading from said analyzer or a second analyzer as a result of a different background gas.

Thus, the present invention may determine whether there is at least one matrix effect exiting in the gaseous sample analysis by comparing the data including the reading and the equal-molar concentrations of the first analyte from two matrixes, one of them is calibrated against and the second one is not. If there is no matrix effect, the data including the reading and/or the concentrations from the two analyzers should be the same to each other. On the other hand, the difference in the reading or the concentrations from the two matrixes indicates the existence of the matrix effect.

For example, if the analyte of a given concentration was introduced to the same analyzer under identical conditions but a different background gas, yields the different reading, the matrix effect exits. For example, 1% Methane having Helium as the background gas yields a reading of 1.2 mV in the first analyzer and 1% Methane having Argon as the background gas yields a reading of 2.4 mV in the same analyzer under the identical conditions. The difference in the reading is caused solely by the different background gases. Thus the matrix effect exits.

Alternatively, the existence of a matrix effect may be included in an analyzer manufacturers' literature. Thus, instead of receiving data and calculating the difference, the existence of the matrix effect could be manually determined and set.

If there is no matrix effect determined, the apparatus then collects data from all the analyzers, stores it in the storage device, and displays it via the display device.(3&4) The data includes but not limited to: the reading and the concentration of each of the analytes in the gaseous sample. The data may be populated using a data collecting and processing software including species of the gaseous sample, analyzers used, times, dates, operators, lot information etc.

If there is at least one matrix effect, a next step is to determine whether the matrix effect can be corrected by applying the correction factor. (5)

If there is the matrix effect wherein linear calibration curves in different background gases have the same slope, the correction factor may be applied. (6) The linear calibration curves and the matrix equation of the second matrix are tested and defined prior to the gaseous sample analysis using methods known in the art, or alternatively, are provided by the manufacturers' literature or other reference available in the art.

If the matrix effect could be corrected by the correction factor, the calculation device then will apply the correction factor to correct the matrix effect. The correction factor may be applied to the reading from the second matrix to obtain a second converted reading of the second matrix. The converted second reading is then introduced to the first matrix equation to obtain a converted second concentration.

Examples of said application have been described in the background of this present application. Then, the apparatus collects the data after the application of the correction factor, stores in its storage device, and displays it via its display device. (4) The data may be populated using data collecting and processing software known in the art.

If the matrix effect could not be corrected by the correction factor since the linear calibration curves from the first and the second matrixes have different slopes, the calculation device will perform linear transformations by applying the correction equation. (7) Then, the apparatus collects the data after the application of the correction factor, stores in its storage device, and displays it via its display device. (4)

The linear transformation takes the reading from the second matrix and converts it into the reading from the first matrix, using a converting equation also known as the correction equation herein.

For example, a first three-point calibration is performed in a first analyzer to establish the first linear calibration curve and the first matrix equation for the first matrix. Other methods of calibration, well known in the art or provided by the manufacturers of the analyzers, could also be applied.

Thus, a typical linear matrix equation could be established and represented as X1=aY1+A, wherein Y1 is a first reading from the first matrix, X1 is a first corresponding concentration of a contaminant in the gaseous sample, a is a first slope of the first Matrix equation, and A is a first intercept of the first matrix equation.

Thus, after the first calibration, the first matrix curve and equation is established in the calculation device. Because the gaseous samples carried by the same matrix shall have the same matrix effect under same conditions such as temperature and pressure, once the first calibration is done and the conditions remain the same, no further calibration shall be necessary for the same matrix in the same analyzer. Data such as the first reading from the first analyzer for the gaseous sample will be stored in the storage device, and the calculation device shall apply the first matrix equation to the reading and obtain the first corresponding concentration of the contaminant.

If, however, the matrix effect is existed in the second matrix, instead of performing calibration using methods described hereabove or otherwise well documented in the art, the present invention largely eliminates a second calibration step to the second matrix.

For example, a second calibration curve and a second matrix equation for the second matrix have been defined prior to the gaseous sample analysis by methods described hereabove. The second matrix equation is established as X2=bY2+B, wherein Y2 is a second reading from the second matrix, X2 is a second corresponding concentration of the contaminant, b is a second slope of the second Matrix equation, and B is a second intercept of the second matrix equation.

Thus, under the same conditions, a first correction equation may be established based on the first and the second matrix equations, which transforms the second reading into the converted second reading. The converted second reading is then introduced to the first matrix equation, and the converted second concentration of the contaminant is obtained.

Because the first correction equation is not subject to change under the same conditions, according to the present invention, the first calibration of the first matrix is all what is needed for the calibration process for the gaseous sample analyses. Even if the matrix effect exits, there is no need to perform the second calibration each and every time. Instead, the calculation device will apply the first correction equation and the first matrix equation to the second reading, and obtain the second converted concentration.

It is to be noticed that the first and the second matrixes could be tested in the same analyzer or different analyzers.

If a second matrix effect exits, a second correction equation is established between the first matrix equation and a third matrix equation using the method described hereabove.

Thus, according to one embodiment of the present invention, if the apparatus determines that there is at least matrix effect exists for the gaseous sample, and at least one matrix effect could not be corrected by the correction factor, the calculation device will apply the correction equation which is pre-set and pre-programmed in the apparatus, and transforms the readings from one matrix to another. An detailed example is described hereafter as EXAMPLE I. It is to be noticed that the EXAMPLE I merely serves as an illustration of one embodiment of the present invention, it should not be viewed in any way a restriction to the present invention.

In one embodiment, the first matrix may not have a matrix effect at all. Thus, the second matrix effect is determined against and converted to the reading of a matrix that does not have the matrix effect. Under this circumstance, the same principle as described herein may still apply.

It is to be noticed that the correspondence between the readings from the first and the second matrixes and the correctness of the correction equation may be subject to change upon the change of the conditions. The conditions may include but not limited to the temperature, the pressure, the altitude, or the humidity. Thus, it is, preferred that the apparatus to be kept and the gaseous sample analysis to be performed in a stable environment. Typically, a lab provides the stable environment so that the transformation equation may remain unchanged. However, if part or whole of the apparatus is subject to relocation where the conditions may be subject to change, the transformation equation may be subject to revisions. Alternatively, the conditions may be input into the correction equation as variations, thus even if the condition is subject to change, it is still not necessary to re-establish or revise the correction equation, but simply inputting the changed conditions into the correction equation.

Therefore, according to the present invention, it eliminates the requirement for the repeated calibration processes. Instead of calibrating each and every matrix in each and every analyzer, the present invention allows one calibration to one matrix in one analyzer for the gaseous sample analysis. The correction equations may be pre-set and pre-programmed in the apparatus and apply to the readings from the analyzers directly in the absence of additional calibrations. This feature is particularly advantageous when multiple analyzers having multiple matrix effects are involved in the gaseous sampling analysis. Because it reduces the calibration process which sometimes operated manually, it may help achieve a higher level of automation even for the low-cost solutions. Because the present invention may eliminate much of the calibration process which usually requires manual operations, it may be an ideal solution for remote operations. For example, the analyzers may be placed at different locations and connected to the apparatus via a means of wireless connections. Only one analyzer, probably located in a convenient location, should be calibrated before the gaseous sample analysis and the rest of the analyzers may send their readings to the apparatus without calibrations. The apparatus then will apply correction factors or correction equations to the readings to obtain the transformed concentrations of the gaseous samples.

### EXAMPLE I

Applying the correction factor in the gaseous sample analysis

The apparatus consisted of connecting cables and a computer system. The connecting cables served as a mean of connection between the apparatus and two analyzers used in this experiment. The computer system comprised at least a hard drive as the storage device, a CPU as the calculation device and a screen as the display device. The experimental gaseous sample test was carried out under a stable environment where the temperature and the pressure remain stable.

Contaminant data at various concentrations in two separate matrix gases was collected. The contaminant in this experiment is Methane. The first matrix gas was Nitrogen; and, the second matrix gas was Helium. The analyzer used in this experiment was Baseline Mocon 9000 Total Hydrocarbon Analyzer.

The contaminant was first analyzed and the readings were obtained under Nitrogen matrix under conditions of the room temperature and atmospheric pressure.

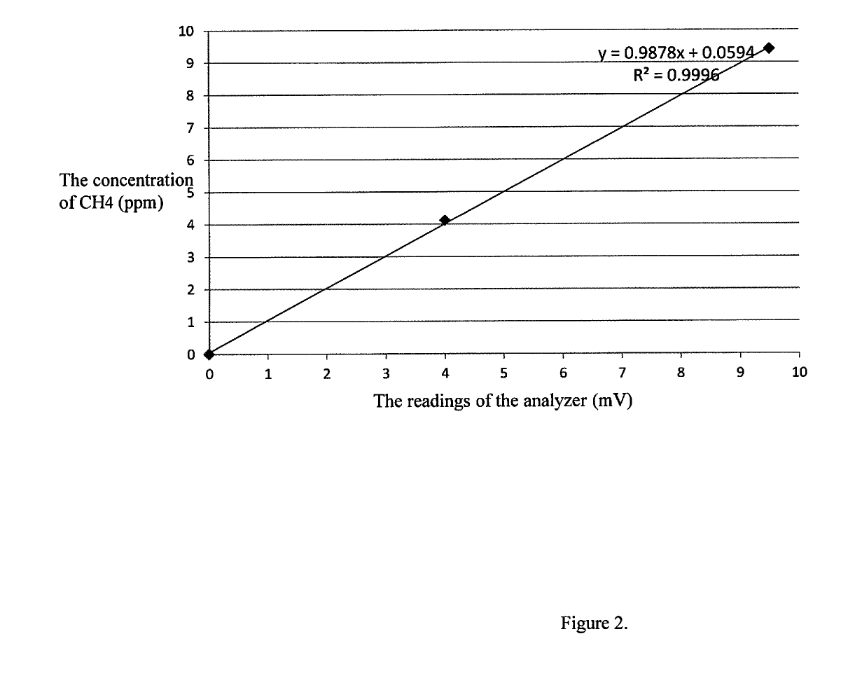

The first analyzer was first calibrated using a calibration method provided by its manufacturer's manual. Calibration samples of Methane with standard contaminant concentrations were provided. Said calibration samples were provided to the first analyzer. Data from each of the calibration samples were collected and stored in the storage device. The first calibration curve and the first matrix equation were established from the calibration as shown in FIG. 2.

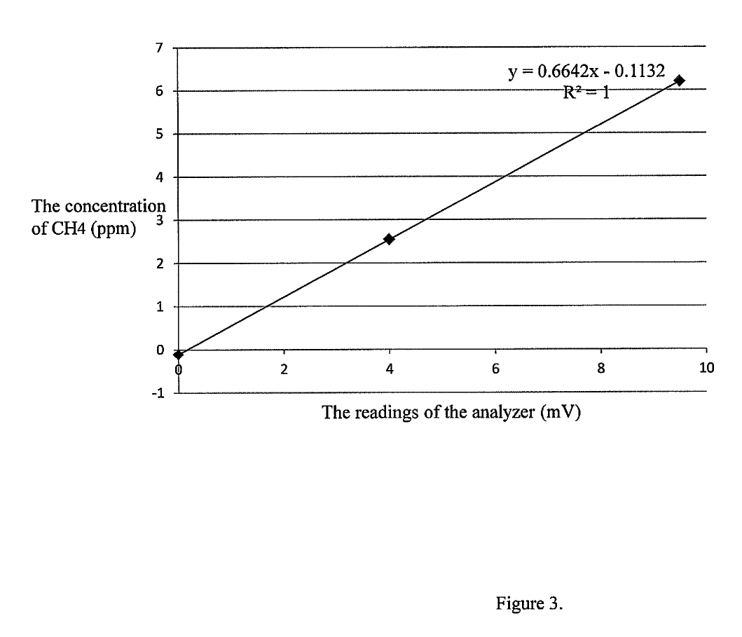

The second calibration curve and the second matrix equation with Helium as the background gas were obtained using the method described hereabove. The results are shown in FIG. 3.

Based on the two matrix equations, the first correction equation was established as: y=(1.48x−0.1675)+0.0594. Where x was the reading from the second matrix, and y was the second converted concentration of the contaminant. The first matrix equation, the second matrix equation, and the first correction equation were then stored in the computer system and might be applied by the calculation device.

The Methane samples having different concentrations were subject to analysis under the first matrix. The samples have containment's concentration at 0, 1, 2, 3, 4, 5, 6, and 7/ppm, respectively. A first set of readings from the samples were collected and stored. The calculation device applied the first matrix equation to the readings and calculates the corresponding concentrations. The concentrations calculated by the first matrix equation were according to the samples' concentrations. The concentrations and the corresponding readings were shown in Table 1.

The Methane samples were then subject to the analysis under the second matrix. A second set of readings were obtained and stored. The calculation device applied the correction equation to the second set of readings to obtain the second converted readings and subsequently, the first matrix equation to the second converted readings to obtain the second converted concentrations as shown in Table 2.

It is to be understood that the use of “including”, “comprising” or “having” and variations thereof herein is meant to encompass the items listed thereafter and equivalents thereof as well as additional items; the terms “a” and “an” herein do not denote a limitation of quantity, but rather denote the presence of at least one of the referenced item; and, the use of terms “first”, “second”, and “third”, and the like, herein do not denote any order, quantity, or importance, but rather are used to distinguish one element from another.

It is to be understood that the above embodiments and examples are provided as illustrations only, and do not in any way restrict or define the scope of the present invention. Various other embodiments may also be within the scope of the claims.

What is claimed is:

1. an apparatus for a gaseous sample test utilizing at least one analyzer, comprising:
(A) a means of connection, wherein said connection may transmit data in between the apparatus and the analyzer;
(B) a storage device;
(C) a calculation device; and,
(D) a display device.

2. The apparatus of claim 1, wherein the storage device, the calculation device and the display device are part of a computer system.

3. A method of simplifying a calibration process of the gaseous sample test utilizing at least one analyzer, comprising:
(A) calibrating a first matrix;
(B) collecting a first reading of a first analyte from the gaseous sample from the first matrix;
(C) collecting a second reading of the first analyte from the gaseous sample from a second matrix;
(D) comparing the first reading and the second reading;
(E) determining a matrix effect exiting if the first reading and the second reading are different.

4. the method of claim 3, further comprising:
(E) after (D), applying a correction factor to the second reading.

5. the method of claim 3, further comprising:
(F) after (D), applying a correction equation to the second reading.

6. A method of obtaining a concentration in a gaseous sample analysis wherein a matrix effect exiting in between a first matrix and a second matrix, comprising:
(A) determining a first matrix equation of the first matrix;
(B) determining a second matrix equation of the second matrix;
(C) establishing a correction equation based on the first and the second matrix equations, wherein the correction equation converts a second reading from the second matrix into a second converted reading;
(D) introducing the second converted reading into the first matrix equation to obtain a second converted concentration.

7. The method of claim 6, wherein the first and second matrixes could be air, Nitrogen, Argon or Helium.
